(19) United States
(12) Patent Application Publication
(10) Pub. No.: US 2022/0005562 A1
(43) Pub. Date: Jan. 6, 2022

(54) MEASUREMENT-FACILITATING DEVICE, METHOD, AND NON-TRANSITORY RECORDING MEDIUM THAT RECORDS PROGRAM

(71) Applicant: OMRON HEALTHCARE Co., Ltd., Kyoto (JP)

(72) Inventors: Hiroshi OGAWA, Kyoto (JP); Toru DENO, Kyoto (JP); Miyuki KIMURA, Kyoto (JP); Takanobu YAMAUCHI, Kyoto (JP); Masaki YAMADA, Kyoto (JP); Mizuki FURUTA, Kyoto (JP)

(21) Appl. No.: 17/448,283

(22) Filed: Sep. 21, 2021

Related U.S. Application Data
(63) Continuation of application No. PCT/JP2020/008794, filed on Mar. 3, 2020.

(30) Foreign Application Priority Data
Mar. 25, 2019 (JP) 2019-056052

(51) Int. Cl. G16H 10/60; G16H 50/30

(52) U.S. Cl. CPC G16H 10/60; G16H 50/30

(57) ABSTRACT

To prompt measurement of corresponding biometric data according to a variation of environmental data. A measurement-facilitating device includes a first acquisition unit that acquires the environmental data regarding an external environment of a user, a first determination unit that determines whether the environmental data meets a preliminarily stored condition regarding an environment set based on the biometric data of the user and indicating that the biometric data of the user is susceptible to a change, and an instruction unit that instructs the measurement of the biometric data when the environmental data is determined to meet the condition.

### CROSS-REFERENCE TO RELATED APPLICATIONS

This application is the U.S. national stage application filed pursuant to 35 U.S.C. 365(c) and 120 as a continuation of International Patent Application No. PCT/JP2020/008794, filed Mar. 3, 2020, which application claims priority to Japanese Patent Application No. 2019-056052, filed Mar. 25, 2019, which applications are incorporated herein by reference in their entireties.

### TECHNICAL FIELD

This invention relates to a measurement-facilitating device, a method, and a non-transitory recording medium that records a program.

### BACKGROUND ART

Recently, performances of medical measurement instruments for household use have been improved, and allows accurately measuring various kinds of biometric data of users and acquiring the measured biometric data by, for example, smartphones for ease of reference for the users. As this medical measurement instrument, for example, a large number of downsized portable electrocardiographs, blood pressure meters, and heart rate meters that can be carried have been sold. Users can easily obtain these instruments and acquire and refer to their own biometric data while living their daily life.

Users who utilize these instruments may make use of the biometric data to review their daily lifestyle so that each kind of biometric data is in an appropriate range recommended by a professional such as a physician to stay in healthy condition.

In addition, in daily life, when an environment changes, the biometric data varies and a physical condition of the user becomes poor in some cases. For example, in a case where a room temperature in a toilet or a dressing room of a bathroom is significantly lower than room temperatures at other rooms, especially in the winter season, in a case where the user goes to a toilet or a dressing room from a warm room, the air temperature difference rapidly increases the blood pressure of the user in some cases.

For example, Patent Document 1 discloses that a symptom level of each disease is predicted based on a weather forecast, and a health warning is transmitted to a member whose symptom level is predicted to be worse.

### CITATION LIST

### Patent Literature

Patent Document 1: JP 2005-50212 A

### SUMMARY OF INVENTION

### Technical Problem

In Patent Document 1, only a disease level is predicted according to the weather forecast, and therefore data related to an environment other than the weather forecast is not referred to. Thus, for example, an influence of indoor environmental changes on the biometric data cannot be considered. In addition to the weather forecast, determination of the environment by which the biometric data of the user is affected and utilization of values according to changes in environmental conditions and biological data, such as a blood pressure, to make use of them for maintaining the health of the user are not performed.

The present invention has been made focusing on the circumstances, and in one aspect, provides a measurement-facilitating device, a method, and a non-transitory recording medium that records a program that prompt a user to measure corresponding biometric data according to a variation of environmental data when an environment of the user changes.

### Solution to Problem

In order to solve the problems described above, the present disclosure employs the following configurations.

That is, a measurement-facilitating device according to a first aspect of the present disclosure includes a first acquisition unit, a first determination unit, and an instruction unit. The first acquisition unit acquires environmental data regarding an external environment of a user. The first determination unit determines whether the environmental data meets a preliminarily stored condition regarding an environment set based on biometric data of the user and indicating that the biometric data of the user is susceptible to a change. The instruction unit instructs measurement of the biometric data when the environmental data is determined to meet the condition.

In the configuration, the measurement-facilitating device acquires the environmental data regarding the external environment of the user (that is, an ambient environment surrounding the user). The environmental data may be any physical amount related to the external environment, and examples of which include a temperature, a humidity, an atmosphere pressure, a wind speed, a brightness, an ambient sound volume, and a cleanliness of air. The first determination unit determines, in some way, whether the biometric data of the user is susceptible to an influence of the change in the environmental data. As long as related to a living body, the biometric data may be any data, and includes blood pressure data, electrocardiographic data, and heart rate data. The first determination unit determines whether the change in the environmental data affects the user for each user. There are many determination methods and several examples are given in the following description of aspects. In a case where the first determination unit determines that the preliminarily stored condition regarding the environment set based on the biometric data of the user and indicating that the biometric data is susceptible to the change is met, the instruction unit instructs the measurement of the biometric data. The instruction unit displays, for example, “Measure the blood pressure” on a display screen, for example, to instruct the blood pressure measurement.

Specifically, in the measurement-facilitating device, it is assumed that, for example, the first acquisition unit acquires air temperature data indicating that a current ambient air temperature of the user rapidly (for example, approximately 10 degrees) decreases, and the first determination unit determines that the preliminarily stored condition indicating that the blood pressure data of the user is susceptible to the influence of the change in the temperature is met based on the temperature data. In this case, the instruction unit instructs the measurement of the blood pressure data. In response to the instruction, the user starts the measurement with a blood pressure meter, or a wristwatch blood pressure meter automatically starts the measurement in response to the instruction.

Thus, according to the measurement-facilitating device according to the first aspect of the present disclosure, when the change in the environment in which the user is likely to be affected occurs, it is possible to prompt the user to measure the biometric data corresponding to the change in the environment, and therefore the user can make use of it for health management of the user himself/herself. This measurement-facilitating device can also identify which environmental change affects which biometric data for each user. Thus, the user can quickly measure this biometric data when the change in the environment affecting their own biometric data occurs, thus ensuring obtaining a relationship between the environmental data and the biometric data backed up by their own actual biometric data.

Furthermore, when the environmental data related to the external environment of the user is acquired, whether the environmental data meets the preliminarily stored condition regarding the environment set based on the biometric data of the user and indicating that the biometric data of the user is susceptible to the change is determined, and the environmental data is determined to meet the condition indicating that the biometric data is susceptible to the change, the measurement-facilitating device according to the first aspect of the present disclosure only needs to be a device that can execute a program to instruct the measurement of the biometric data, and for example, may be a wearable appliance (e.g., a smartphone, a wristwatch wearable terminal), an activity meter, a stationary device (e.g., a personal computer). Furthermore, for example, the user need not wear the measurement-facilitating device and the measurement-facilitating device only needs to acquire the environmental data. For example, the user may wear a measurement device that can measure or acquire the environmental data, and the measurement-facilitating device may acquire the environmental data (or, for example, the air temperature, which is one of the environmental data) from the device and execute the program described above.

A measurement-facilitating device according to a second aspect of the present disclosure further includes a calculation unit that calculates the condition based on a correlation between the measured environmental data and the measured biometric data.

In the configuration, the measurement-facilitating device calculates the condition based on the correlation between the measured environmental data and the measured biometric data in advance, and thus the conditions can be preliminarily set from the correlation of the measured data for each user in advance. Thus, according to this configuration, the condition is calculated using the actually measured data for each user, so it is possible to calculate the condition in which the characteristic is grasped for each user.

In a measurement-facilitating device according to a third aspect of the present disclosure, the calculation unit includes: an association unit that associates an amount of variation of the measured environmental data with an amount of variation of the measured biometric data that varies in synchronization with the amount of variation of the measured environmental data; and a setting unit that sets the amount of variation of the measured environmental data matching the amount of variation of the environmental data acquired by the first acquisition unit within a certain range as meeting the condition.

In the configuration, the association unit included in the calculation unit associates the amount of variation of the measured environmental data that was measured in the past and the amount of variation of the measured biometric data that varied in synchronization and was measured in the period in which the amount of variation of the measured environmental data measured. For example, in a case where a threshold is set to the amount of variation (also referred to as an amount of change) of the measured biometric data for each type of the biometric data, only the environmental data corresponding to the biometric data that changes by equal to or more than the threshold is focused, and the environmental data whose amount of variation changes by equal to or more than the threshold in conjunction with the biometric data, data of the amount of variation of the measured environmental data and the amount of variation of the measured biometric data are determined to vary in synchronization. The amount of variation of the measured environmental data is associated with the amount of variation of the measured biometric data. Then, the amount of variation of the measured environmental data associated by the association unit matching the amount of variation of the environmental data acquired by the first acquisition unit within a certain range can be set as meeting the condition. As a result, the data of the amount of variation of the measured environmental data and the amount of variation of the measured biometric data regarded to vary in synchronization and associated are referred to. When the first acquisition unit acquires the environmental data whose amount of variation is similar to the amount of variation of the measured environmental data, the first determination unit determines that the environmental data meets the condition, and the biometric data corresponding to the environmental data is determined to be susceptible to the change in the environment.

Accordingly, according to the measurement-facilitating device according to the third aspect of the present disclosure, the setting unit can set the condition for determining whether the current biometric data of the user is likely to change by the current environmental data from the past measurement data based on the environmental data whose amount of variation is equal to or more than a fixed value (the threshold) among the environmental data and the biometric data measured in the past.

A measurement-facilitating device according to a fourth aspect of the present disclosure further includes a reception unit that receives a content of a change in a physical condition that the user is aware of. The association unit associates the amount of variation of the measured environmental data, the content of the change in the physical condition, and the amount of variation of the measured biometric data based on the content of the change in the physical condition and the amount of variation of the measured biometric data.

In the configuration, when the user is aware of the change in the physical condition, the measurement-facilitating device acquires the content via the reception unit, and the association unit associates the content of the change in the physical condition, the amount of variation of the measured biometric data, and the amount of variation of the measured environmental data, thereby ensuring associating an influence of the environment on the user including the physical condition of the user. Thus, the subjective consciousness of the physical condition of the user, the objective measured biometric data and environmental data can be associated, and therefore the influence on the user due to the variation in the environmental data can be more multidirectionally determined.

A measurement-facilitating device according to a fifth aspect of the present disclosure further includes a third acquisition unit that acquires a table indicating a correspondence with the measured environmental data meeting the condition based on a type of the biometric data and a correlation between the measured environmental data and the measured biometric data. The first determination unit refers to the table to determine whether the environmental data acquired by the first acquisition unit meets the condition.

In the configuration, the table indicating the correspondence between the type of the biometric data and the measured environmental data by which the biometric data of the user is affected (that is, the condition is met) preliminarily created in some way is acquired, the first determination unit refers to the table and determines that the measured environmental data described in the table affects the corresponding biometric data, that is, the acquired environmental data meets the condition. Thus, the first determination unit refers to the table and can determine whether the biometric data of the user is affected by the change in the environment by the environmental data acquired by the first acquisition unit.

Therefore, according to the measurement-facilitating device according to the fifth aspect of the present disclosure, when the measured environmental data corresponding to the table is present, it can be determined that the corresponding biometric data is susceptible to the change influenced by the environment. By simply referring to the table in this way, whether the biometric data is susceptible to the change in the corresponding environment can be easily determined. Since only whether the corresponding environmental data is present in the table is determined, an operation resource of the CPU is expected to be small.

In a measurement-facilitating device according to a sixth aspect of the present disclosure, the table is determined for each user based on an amount of variation of the measured environmental data and an amount of variation of the measured biometric data.

In the configuration, for example, in the case where the amount of variation of the measured environmental data and the amount of variation of the measured biometric data vary in synchronization and associated, it is regarded that the variation in the environmental data affects the variation in the biometric data and are included in the table. In other words, in a case where the amount of variation of the environmental data is equal to or more than a certain amount, when the amount of variation of the biometric data is equal to or more than the fixed value (the threshold) corresponding to the change in the environmental data, it is determined that the biometric data changes by the change in the environmental data, and the measured environmental data is associated with the measured biometric data and the associated data are included in the table. Furthermore, the amount of variation of the environmental data possibly depends on a value (for example, a value before the variation), and therefore, not only the amount of variation but also the value may be included in the table for determination. Furthermore, since the amount of variation of the biometric data possibly changes depending on the value, the threshold of the amount of variation may be changed according to the value.

Thus, according to the measurement-facilitating device according to the sixth aspect of the present disclosure, the table can be set for each user based on the amounts of variation of the actually measured environmental data and the measured biometric data. Since the table is set based on the actual data for each user, the table true of the user can be created.

A measurement-facilitating device according to a seventh aspect of the present disclosure further includes a second determination unit that determines whether the user is indoors. The first acquisition unit changes the acquired environmental data by whether the user is outdoors or indoors.

In the configuration, the second determination unit determines whether the user is outdoors or indoors, and the environmental data that affects the user differs between outdoors and indoors, so the first acquisition unit changes the acquired environmental data by whether the user is outdoors or indoors. For example, in a case where the first acquisition unit acquires position information of the user indicating that the user is outdoors, the first acquisition unit acquires outdoor data, such as an outside temperature and external humidity, via, for example, the Internet. When the position information of the user indicates that the user is indoors, indoor data measured by, for example, an air temperature sensor or a humidity sensor installed indoors may be acquired via a near-field wireless communication. The first determination unit may change the environmental data used by which of the outdoor environmental data and the indoor environmental data is acquired by the first determination unit and whether the user is outdoors or indoors.

In a measurement-facilitating device according to an eighth aspect of the present disclosure, when the user is determined to be indoors, the first determination unit acquires the environmental data from a sensor installed indoors based on a near-field wireless communication, preferentially employs the indoor environmental data over the outdoor environmental data, and determines whether the biometric data of the user is susceptible to a change in an environment.

In the configuration, when the first determination unit determines that the user is indoors, the first determination unit determines using the environmental data acquired by, for example, the sensor set indoors. As such, the first determination unit can change the environmental data employed depending on a location of the user, and it is possible to grasp a variation in the environmental data that accurately reflects an external environment of the user.

In a measurement-facilitating device according to a ninth aspect of the present disclosure, when the first acquisition unit determines that the acquired environmental data meets the condition, the instruction unit notifies the user of an alert indicating that the biometric data is likely to change by an environment.

In the configuration, when the first acquisition unit determines that the acquired environmental data meets the condition (the biometric data of the user is susceptible to the change in the environment), the instruction unit alerts the user to the fact for calling attention. Thus, the user can measure own biometric data and can record the biometric data according to the change in the environment.

In a measurement-facilitating device according to yet another aspect of the present disclosure, the reception unit displays an interface for allowing the user to execute for reception of the content that the user is aware of.

In the configuration, the reception unit in the measurement-facilitating device displays the interface for the user to receive the content of the change in the physical condition that the user is aware of, and the user can use this interface to input the content of the change in physical condition to the measurement-facilitating device. As long as the user can input data, any interface may be used, and example of the interface includes a button of a Graphical User Interface (GUI). For example, the user selects one or more buttons from a plurality of buttons in the GUI to perform the input to the measurement-facilitating device.

### Advantageous Effects of Invention

According to the measurement-facilitating device, the method, and the program of the present invention, in one aspect, when the change in the environment to which the user is susceptible occurs, the user is prompted to measure the biometric data corresponding to the change in the environment and the influence of the environmental change on a living body of the user can be determined.

### BRIEF DESCRIPTION OF DRAWINGS

FIG. 1 is a diagram illustrating an overview of a system including a measurement-facilitating device, a wristwatch wearable terminal, and a server connected over a network according to an embodiment.

FIG. 2 is a drawing schematically illustrating an example of a hardware configuration of the measurement-facilitating device according to an embodiment.

FIG. 3 is a drawing illustrating an example of a part of a software configuration of the measurement-facilitating device according to an embodiment.

FIG. 4 is a flowchart schematically depicting an example of a processing procedure regarding the measurement-facilitating device according to an embodiment.

FIG. 5 is a flowchart schematically depicting an example of a processing procedure of a table creation regarding the measurement-facilitating device according to an embodiment.

### DESCRIPTION OF EMBODIMENTS

An embodiment according to an aspect of the present invention (hereinafter, also referred to as “the present embodiment”) will be described below with reference to the drawings. Note that, in the following embodiment, parts denoted by the same reference numbers perform the same operations, and a repetitive explanation will be omitted.

### Overview

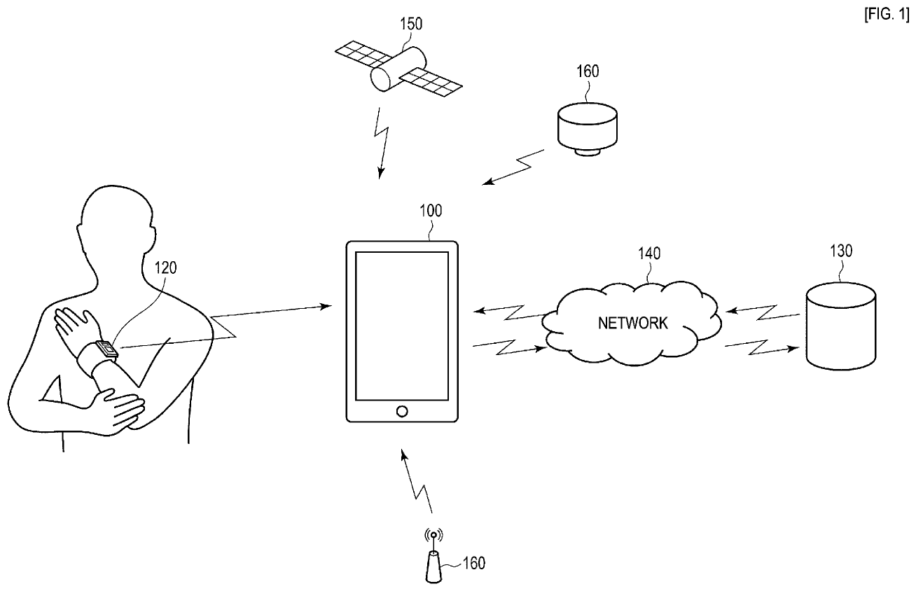

First, with reference to FIG. 1, the overview of a measurement-facilitating device of the present invention will be described.

FIG. 1 schematically illustrates an example of a measurement-facilitating device 100, a wristwatch wearable terminal 120, a server 130, a network 140, a GPS satellite 150, and a sensor 160 according to an example of the overview.

The measurement-facilitating device 100 receives environmental data directly from the sensor 160 installed outdoors and/or indoors, or acquires the environmental data from the server 130 acquiring and storing the environmental data over the network 140. The measurement-facilitating device 100 determines whether there is a possibility that biometric data of a user varies according to a variation in the environmental data in a case where the environmental data changes. When it is determined that there is a possibility of the variation in the biometric data of the user, the measurement-facilitating device 100 instructs the user to measure the corresponding biometric data. For example, when the measurement-facilitating device 100 determines whether the acquired environmental data meets a preliminarily stored condition regarding the environment set based on the biometric data of the user and indicating that the biometric data of the user is susceptible to the change in the environment. When the measurement-facilitating device 100 determines that the acquired environmental data meets this condition, it is determined that there is a possibility of the variation in the biometric data of the user, and the measurement-facilitating device 100 instructs the user to measure the biometric data corresponding to the environmental data.

The measurement-facilitating device 100 acquires own position data in some way, determines whether to acquire outdoor environmental data or indoor environmental data based on the position data, and acquires the desired indoor or outdoor environmental data where the user is located. For example, the measurement-facilitating device 100 receives signals from a plurality of the GPS satellites 150 and calculates the position of the measurement-facilitating device 100. Not only receiving the signals from the GPS satellites 150 and calculating the position information of the user by the measurement-facilitating device 100, the measurement-facilitating device 100 may receive data regarding the position from a base station and/or a wireless LAN access point and correct the position information according to the data.

When the measurement-facilitating device 100 determines that the biometric data varies, the measurement-facilitating device 100 outputs an instruction to measure the biometric data. For example, when the user receives this instruction, the user may use the wristwatch wearable terminal 120 and press, for example, a button to measure the corresponding biometric data to start the measurement. The measurement-facilitating device 100 may receive the measured biometric data, associate the biometric data with the environmental data, and store it. The biometric data associated with this environmental data may also be stored in the server 130 over the network 140.

The wristwatch wearable terminal 120 is worn (or possessed) by the user, measures the biometric data of the user through the operation by the user, and transmits the biometric data to the measurement-facilitating device 100. As long as related to a living body, the biometric data may be any data, and examples of which include electrocardiographic data, blood pressure data, and heart rate data. The biometric data may be set to be measured based on, not only the operation of the user, but also a certain event.

The server 130 connects to and exchanges data with the measurement-facilitating device 100 over the network 140. The server 130 receives a request for a certain type of the environmental data from the measurement-facilitating device 100, and transmits the corresponding environmental data to the measurement-facilitating device 100 over the network 140. The server 130 acquires various kinds of environmental data from, for example, sensors, such as meteorological sensors set at various locations, and enterprises dealing with environmental data and stores them.

The environmental data stored by the server 130 may be any data as long as the data related to the environment. Examples of the environmental data include an air temperature, an atmosphere pressure, a humidity, a force velocity, a brightness, a sound volume (can be acquired by a smartphone or a smartwatch), a cleanliness of air (air quality index (mass per unit volume of PM2.5, NO2, O3, SO2, PM10, CO, etc.)). The data of the air temperature, the atmosphere pressure, the humidity, and the force velocity can be acquired from, for example, weather sites and weather-related business operators. The data of the brightness can be acquired by a sensor that can measure brightness of a smartphone. The sound volume is, for example, a noise around the user, and can be measured using a microphone of, for example, a smartphone and/or a smartwatch. Additionally, a noise is measured by, for example, a municipality, especially in an urban area, and therefore the sound volume data (noise data) may be acquired from the municipality. The index of air quality can be associated with the cleanliness of air. Examples of the air quality index include the mass per unit volume of PM2.5, NO2, O3, SO2, PM10, and CO. As these values become small, the air can be determined as clear.

Note that in a case where the measurement-facilitating device 100 alone can measure the biometric data measured by the wristwatch wearable terminal 120, the biometric data measured by the measurement-facilitating device 100 may be used. In this case, the measurement-facilitating device 100 includes a device portion that calculates the biometric data of the wristwatch wearable terminal 120. For example, when the measurement-facilitating device 100 is a wearable terminal device (for example, a smartphone), the measurement-facilitating device 100 usually includes a photographing device (such as a camera) and a lighting device, and the heart rate can be measured, so the heart rate data can be calculated.

As described above, when the wristwatch wearable terminal 120 acquires the environmental data of the user and various kinds of the environmental data vary, the measurement-facilitating device of the present embodiment determines whether the biometric data of the user varies. When the measurement-facilitating device determines that the biometric data varies, the measurement-facilitating device 100 instructs the measurement of the biometric data. As a result, when the environment changes for the user, what change occurs in which biometric data due to the environmental change can be measured. Thus, since the data indicating that a change appears in which biometric data by which environmental change can be acquired for each user, which environmental change needs to cope with can be found for each user, and making use of the data allows maintaining the health.

### Configuration Example

### Hardware Configuration

### Measurement-Facilitating Device

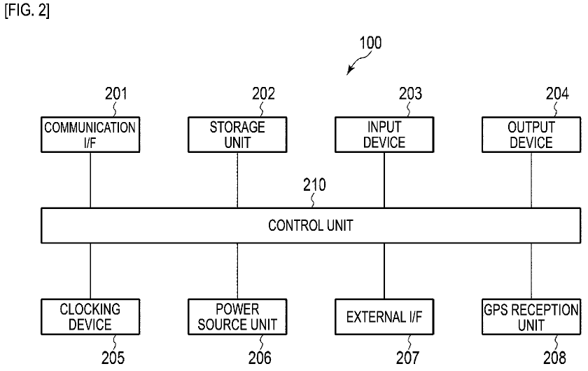

Next, an example of the hardware configuration of the measurement-facilitating device 100 according to the present embodiment will be described using FIG. 2.

As illustrated in FIG. 2, the measurement-facilitating device 100 according to the present embodiment includes a computer to which a communication interface 201, a storage unit 202, an input device 203, an output device 204, a clocking device 205, a power source unit 206, an external interface 207, a GPS reception unit 208, and a control unit 210 are electrically connected. The measurement-facilitating device 100 according to the present embodiment is equivalent to the “measurement-facilitating device” of the present invention. Note that, in FIG. 2, the communication interface and the external interface are described as “communication I/F” and “external I/F”, respectively.

The communication interface 201 is an interface for wired or wireless communications over a network, such as a near-field wireless communication (for example, Bluetooth (registered trademark)) module, a wired local area network (LAN) module, and a wireless LAN module. The communication interface 201 is an interface for connecting the measurement-facilitating device 100 to an external device (for example, a computer and communication equipment on the network; the wristwatch wearable terminal 120, the server 130, and the sensor 160 in the example of FIG. 1). The communication interface 201 is controlled by the control unit 210 and is to, for example, receive the environmental data from the sensor 160 and receive the biometric data from the wristwatch wearable terminal 120. In addition, the communication interface 201 downloads the environmental data from the server 130 over the network 140. Note that the short-range wireless communication system is not limited to a special method, may be any communication method, and examples of which include Bluetooth (registered trademark) and Near Field Communication (NFC).

The communications over the network are usually wireless, but may be wired. The network may be an internetwork including the Internet, another type of a network, such as an intra-hospital LAN, or one-to-one communications using, for example, a Universal Serial Bus (USB) cable. The communication interface 201 may include a micro USB connector.

The storage unit 202 is a medium that accumulates, for example, information about a recorded program by electrical, magnetic, optical, mechanical, or chemical action such that, for example, the information about the program can be read by, for example, any device including a computer and a machine. The storage unit 202 is, for example, an auxiliary storage device, such as a hard disk drive and a solid state drive. The storage unit 202 stores the environmental data acquired from the server 130 and/or the sensor 160 (may also include whether the environmental data is acquired indoors) and the biometric data acquired from the wristwatch wearable terminal 120. Furthermore, the storage unit 202 also stores physical condition data of the user input to the input device 203 by the user. Furthermore, the storage unit 202 stores a preliminarily stored condition regarding the environment set based on the biometric data of the user and indicating that the biometric data of the user is susceptible to the change in the environmental data. This condition is, for example, that an amount of variation of the measured biometric data that varies in synchronization with an amount of variation of the measured environmental data is associated with the amount of variation of the environmental data, and the amount of variation of the measured environmental data matches the amount of variation of the acquired environmental data within a certain range.

Furthermore, the storage unit 202 may also store determination data indicating whether the environmental data is determined to affect the biometric data of the user. The actual biometric data, environmental data, and determination data stored in the storage unit 202 may be determined for each user to adjust a threshold for the determination by the control unit 210 to increase the determination accuracy, or learning may be performed based on the data.

Furthermore, the storage unit 202 may transmit the biometric data, the environmental data, and the determination data, and the server 130 may store the data for each user, further separately acquire personal data of the user (also referred to as attribute data that includes, for example, an age, body information (such as a height and a weight), a medical history, and a hospital visit record), and collect the considerably enormous quantity of data. Based on the data, acquisition of features of data, calculation of a highly accurate threshold for determination, and discovery of a relationship between the environmental data and the biometric data for each user may be executed by using the data.

Furthermore, the storage unit 202 stores an execution program. The execution program determines whether the biometric data of the user is susceptible based on the acquired environmental data performed in the control unit 210, and instructs the user to measure the biometric data when the biometric data of the user is determined to be susceptible to the environment.

Furthermore, the storage unit 202 may store all of the environmental data acquired from the server 130 and/or the sensor 160 and the biometric data acquired by the wristwatch wearable terminal 120 corresponding to the environmental data for a predetermined period. The predetermined period may be, for example, a certain period in the past, or up to a certain future date and time including the past to the present. Furthermore, in a case where the storage unit 202 includes the past environmental data and the corresponding biometric data prior to the determination by the control unit 210, the measurement-facilitating device 100 can determine whether the measured environmental data brings a change in the biometric data of the user based on the data. Besides, the storage unit 202 stores the physical condition data related to the physical condition of the user received by the input device 203 along with the date and time data and associated with the biometric data and the environmental data.

The input device 203 is a device that receives inputs, and examples of which include a touch panel, a physical button, a mouse, and a keyboard. The input device 203 receives, for example, the physical condition data of the user. The output device 204 is an output device that performs outputs, and outputs information by, for example, display, sound, paper medium, light, and vibration, and examples of the output device 204 include a display, a speaker, and a printer. Information can be input to the touch panel by touching the display panel with, for example, a finger and a pen, and the touch panel displays buttons to be touched and other pieces of information and thus is compatible with both the input device 203 and the output device 204. For example, the output device 204 displays the instruction content from the control unit 210 and transmits it to the user by, for example, sound, light, or vibration.

The clocking device 205 is a device that measures time, and can measure a date and a time. For example, the clocking device 205 may be a clock that includes a calendar, and passes the current year, month, and/or date and time information to the control unit 210. With the date and time of this clocking device 205, a time stamp is given to data, and the data can be stored by the storage unit 202.

As long as power can be supplied, any power source unit 206 can be used, and examples of which include a chargeable secondary battery or an AC power source that can be acquired from a usual outlet. The power source unit 206 supplies power to each element mounted on the body of the measurement-facilitating device 100. The power source unit 206 supplies power to, for example, the communication interface 201, the storage unit 202, the input device 203, the output device 204, the clocking device 205, the external interface 207, the GPS reception unit 208, and the control unit 210.

The external interface 207 is for mediating between the body of the measurement-facilitating device 100 and its exterior, for example, a USB port, and is an interface for connection with an external device (e.g., a memory, communication equipment, a biometric data measurement device). The external interface 207 is an interface for connection with an external device, such as an electrocardiograph, a blood pressure meter, a pedometer, an activity meter, and/or an acceleration sensor.

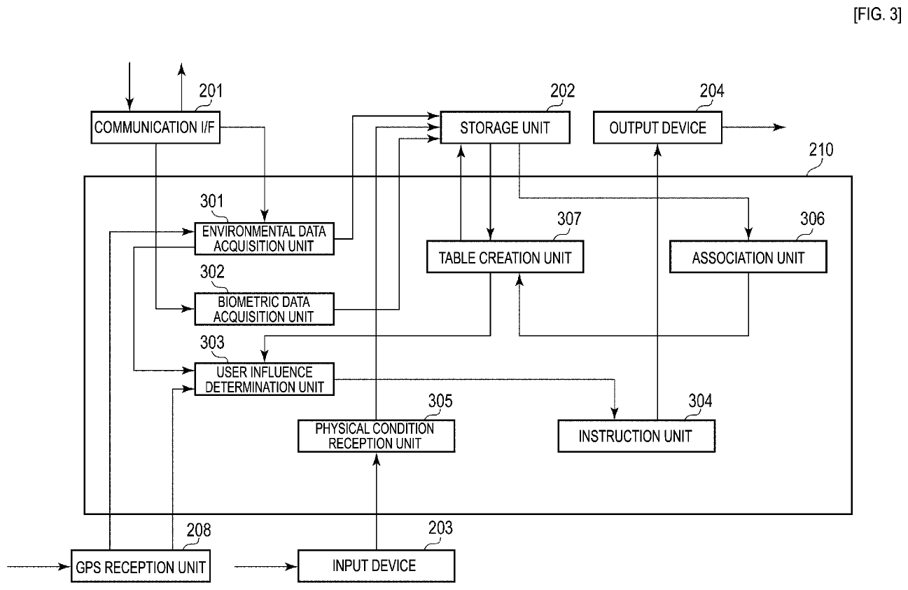

The control unit 210 includes, for example, a central processing unit (CPU), a random-access memory (RAM), and a read only memory (ROM), and controls each constituent element in accordance with information processing. The storage unit 202 stores the execution program that determines whether the biometric data of the user varies based on the variation in the acquired environmental data, and the control unit 210 invokes the execution program from the storage unit 202 and executes the processing. Details of the control unit 210 will be described with reference to FIG. 3.

### Software Configuration

### Measurement-Facilitating Device 100

Next, an example of the software configuration of the measurement-facilitating device 100 according to the present embodiment will be described using FIG. 3. FIG. 3 illustrates the software configuration in which the control unit 210 in the measurement-facilitating device 100 acquires the environmental data and determines whether the biometric data of the user varies in association with the variation of the environmental data, and when the biometric data is determined to vary in association with the variation of the environmental data, the program to instruct the measurement of the biometric data is executed.

In executing the required program, the control unit 210 in the measurement-facilitating device 100 determines whether the biometric data varies based on the variation in the acquired environmental data stored in the storage unit 202. When the biometric data is determined to vary, the control unit 210 develops the execution program that instructs the measurement of the biometric data (in other words, the execution program that determines whether the acquired environmental data meets the condition indicating that the biometric data of the user is susceptible to the change) in a RAM. The control unit 210 interprets and executes the execution program unfolded in the RAM by the CPU, and controls each component. Thus, as illustrated in FIG. 3, the measurement-facilitating device 100 according to the present embodiment includes an environmental data acquisition unit 301, a biometric data acquisition unit 302, a user influence determination unit 303, an instruction unit 304, a physical condition reception unit 305, an association unit 306, and a table creation unit 307.

The environmental data acquisition unit 301 acquires the indoor and/or outdoor environmental data from, for example, the sensor 160 and/or the server 130 via the communication interface 201. The environmental data acquisition unit 301 receives signals from the plurality of GPS satellites 150 and receives the position data of the measurement-facilitating device 100 from the GPS reception unit 208, which generates the position data, and determines whether the user who possesses the measurement-facilitating device 100 is indoors. When the user is indoors, the environmental data acquisition unit 301 acquires the indoor environmental data from, for example, the sensor 160, and when the user is outdoors, the environmental data acquisition unit 301 acquires the outdoor environmental data from the server 130 over the network 140. The environmental data acquisition unit 301 may cause the storage unit 202 to store the measured environmental data along with the measured biometric data acquired by the biometric data acquisition unit 302.

Note that the sensors that detect the environmental data include, for example, the sensors 160 installed indoors and outdoors, but the measurement-facilitating device 100 may include these sensors and the environmental data acquisition unit 301 may acquire the environmental data from these sensors.

The biometric data acquisition unit 302 acquires the biometric data of the user measured by, for example, the wristwatch wearable terminal 120 via the communication interface 201. In the present embodiment, when it is determined that the environmental data acquired by the measurement-facilitating device 100 meets the condition, the measurement-facilitating device 100 instructs the user to measure the biometric data, and the biometric data acquisition unit 302 acquires the biometric data measured based on this instruction. The condition is the preliminarily stored condition with which the biometric data of the user is determined to be susceptible to the change in the environmental data. For example, in a case where the measured environmental data correlates with the measured biometric data and the amounts of variation of these pieces of data vary in synchronization, when the amount of variation of the measured environmental data matches the amount of variation of the acquired environmental data within a certain range, the condition is met.

For example, in a case where the biometric data is the electrocardiographic data, the biometric data acquisition unit 302 acquires an electrical potential difference signal, which indicates an electrical potential difference between two electrodes output from a signal processing circuit in the wristwatch wearable terminal 120 in the form of time series data, as the electrocardiographic data via the communication interface 201. Here, the electrocardiographic data is a waveform signal that represents an electrical activity of a heart. In a case where the biometric data is the blood pressure data, the biometric data acquisition unit 302 acquires the blood pressure data measured by the wristwatch wearable terminal 120 (time series data including systolic blood pressure (SBP) and diastolic blood pressure (DBP)) via the communication interface 201.

The biometric data acquired by the biometric data acquisition unit 302 is stored in the storage unit 202. The biometric data acquisition unit 302 may store all measured biometric data along with the measured environmental data in the storage unit 202.

Note that the sensors that detect the external biometric data are, for example, mounted on the wristwatch wearable terminal 120, but the measurement-facilitating device 100 may include these devices, and the biometric data acquisition unit 302 may acquire the biometric data from these devices.

For example, when the biometric data is the electrocardiographic data, the electrocardiographic data is data indicative of a temporal change of electrical excitation of the heart and calming down of the excitation, and the electrical potential changes due to a varying electric field generated on the body surface by the electrical excitation of the heart. When the biometric data is the blood pressure data, the biometric data acquisition unit 302 acquires time series data including the systolic blood pressure (SBP) and the diastolic blood pressure (DBP).

The user influence determination unit 303 receives the environmental data from the environmental data acquisition unit 301, and determines whether the environmental data meets the condition indicating that the biometric data of the user is susceptible to the change in the environment. For example, the user influence determination unit 303 determines whether the environmental data varies with respect to time (hereinafter also referred to as time variation or simply referred to as variation). When the environmental data varies, the user influence determination unit 303 determines whether a possibility that the biometric data of the user is affected is high (also referred to as whether the biometric data is susceptible to the change in the environment). “The biometric data is affected” typically means worsening of the biometric data of the user, and an example of which includes that a certain value of the biometric data is apart from an appropriate range more than a threshold. This appropriate range may be preliminarily stored in the user influence determination unit 303, or may be set for each user and stored in the storage unit 202. Furthermore, this appropriate range may be provided by the table creation unit 307 for each type of the biometric data. The user influence determination unit 303 may determine whether the user is indoors from the GPS reception unit 208, and may change the threshold indicating a degree of the separation from the appropriate range depending on whether the user is indoors.

In a case where the user influence determination unit 303 determines that the environmental data meets the condition indicating that the biometric data of the user is susceptible to the change in the environment, the user influence determination unit 303 instructs the instruction unit 304 to measure the biometric data. For example, in a case where the user influence determination unit 303 determines whether a possibility that the biometric data of the user varies more than the threshold by the time variation of the environmental data is high and determines that the possibility that the biometric data varies is high, the user influence determination unit 303 passes the corresponding biometric data to the instruction unit 304 instructed to measure the biometric data. For example, this condition may be included in a table created by the table creation unit 307 or stored in the storage unit 202. The user influence determination unit 303 determines whether the condition is met with reference to the table and/or the storage unit 202.

The instruction unit 304 receives the instruction data to measure certain biometric data from the user influence determination unit 303, passes the instruction data to the output device 204, and outputs the instruction data in a desired format. The user receives the output instruction data, and the user operates to measure the corresponding biometric data. The measurement result by the user is measured, for example, in the wristwatch wearable terminal 120, and the measured data is received by the measurement-facilitating device 100 via the communication interface 201.

The physical condition reception unit 305 receives the data input when the user feels a disorder of the physical condition via the input device 203, and causes the storage unit 202 to store the data associated with the biometric data and the environmental data. For example, this input is performed as follows. The input device 203 displays an interface. The physical condition reception unit 305 receives the physical condition data indicating the physical condition by, for example, touching any option in the display via the input device 203. For example, the user enters a content, such as stifling, dizziness, and coughing, by the input device 203 and the physical condition reception unit 305. The input device 203 may display these contents so that the user can perform the selection with, for example, a touch panel.

With the measured environmental data and the measured biometric data stored in the storage unit 202 and correlated with one another, the association unit 306 associates the amount of variation of the measured environmental data with the amount of variation of the measured biometric data varies in synchronization with the amount of variation of the measured environmental data. Based on the amount of variation (also referred to as an amount of change) of the measured biometric data corresponding to the content of the physical condition data, the association unit 306 may further associate the measured environmental data correlated with the biometric data, the measured biometric data, and the physical condition data, among the measured environmental data, the measured biometric data, and the physical condition data stored in the storage unit 202. For example, in a case where it can be determined that the content of the physical condition data is associated with the amount of variation of the corresponding measured biometric data, the association unit 306 associates these pieces of data with the measured environmental data. As an example, when the physical condition of the user indicated by the physical condition is associated with the type of the measured biometric data, further the amount of variation of the measured biometric data is greater than or equal to a fixed value, and a time when the measured physical condition data varies and the time when the physical condition reception unit 305 receives the disorder of the physical condition of the user are within a predetermined time, the association unit 306 associates the measured environmental data, the measured biometric data, and the physical condition data. In the association unit 306, for example, the physical condition or a disease condition caused by the variation in the biometric data has been known depending on the type of the biometric data in some cases, and therefore, whether the physical condition of the user indicated by the physical condition data is associated with the type of the biometric data is determined by whether the physical condition or the disease condition corresponds to the known one. Note that the time at which the user has felt the disorder of the physical condition may be settable later by the user via the input device 203, not the time received by the physical condition reception unit 305 from the user. Additionally, the content of the user feeling the disorder also may be settable later via the input device 203.

The table creation unit 307 sets the match of the amount of variation of the measured environmental data associated by the association unit 306 with the amount of variation of the acquired environmental data within a certain range as meeting the condition, and creates a table showing the correspondence between the measured environmental data and the measured biometric data meeting this condition. Furthermore, the table creation unit 307 may associate content of the change in the physical condition that the user is aware of with the amount of variation of the measured environmental data and the amount of variation of the measured biometric data, and include the associated data in the table. Based on the measured environmental data, the measured biometric data, and the physical condition data stored in the storage unit 202, the table showing the correspondence with the measured environmental data that affects the user is created for each type of the measured biometric data. For example, based on the measured environmental data, the measured biometric data, and the physical condition data stored in the storage unit 202, the table creation unit 307 may determine and select the measured environmental data that adversary affects the measured biometric data and/or the physical condition data due to the variation in the measured environmental data and may create the table in which these pieces of the selected data are associated. Whether a negative effect is given to the health condition of the user is determined as follows. For example, in a case where a threshold or a range is set to each data item and the threshold and/or the range is exceeded, it is determined that the negative effect is given. The table created by the table creation unit 307 may be stored in the storage unit 202, and the user influence determination unit 303 may refer to the storage unit 202.

Furthermore, this table is stored in the storage unit 202 in advance, and the table creation unit 307 may acquire the table from the storage unit 202. In this case, the table is determined based on information about the previous biometric data and environmental data.

The table creation unit 307 provides the created or acquired table to the user influence determination unit 303 to determine whether the environmental data acquired by the environmental data acquisition unit 301 affects the health of the user.

### Other

Operations of the measurement-facilitating device 100 will be described in detail in an operation example described later. Note that, in the present embodiment, the control unit 210 in the measurement-facilitating device 100 may be achieved by a general-purpose CPU. Nevertheless, a portion or all of the above operations (or functions) may be achieved by one or a plurality of dedicated processors. Further, the configuration of the measurement-facilitating device 100 may be appropriately omitted, replaced, and added as appropriate according to the embodiment.

### Operation Example: Overall

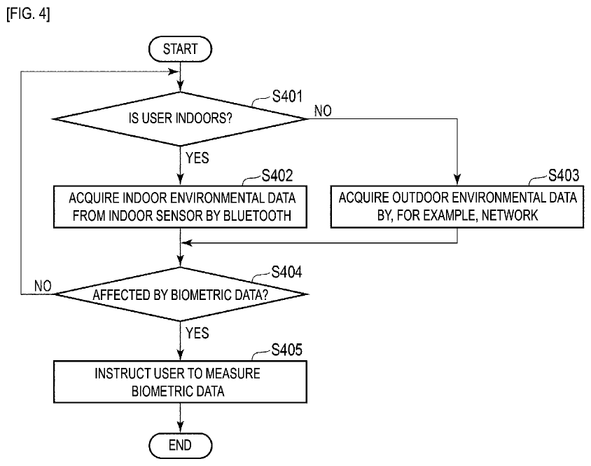

Next, an overview of the operations of the measurement-facilitating device 100 will be described with reference to FIG. 4.

FIG. 4 is a flowchart depicting an example of a processing procedure of the measurement-facilitating device 100. Note that the processing procedure described below is merely an example, and each process may be changed to the extent possible. Further, in the processing procedure described below, steps can be omitted, substituted, and added in accordance with the embodiment as appropriate.

### Activation

First, the user starts the measurement-facilitating device 100 via the input device 203, and further receives inputs, such as a configuration. The control unit 210 in the measurement-facilitating device 100 advances processing according to the following processing procedure.

### Step S401

In step S401, the control unit 210 operates as the environmental data acquisition unit 301, acquires the position data of the measurement-facilitating device 100 from the GPS reception unit 208, and determines whether the position of the measurement-facilitating device 100 (i.e., the position of the user) is indoors or outdoors. When it is determined that the user is indoors, the process proceeds to step S402, and when it is determined that the user is not indoors, the user is determined to be outdoors and the process proceeds to step S403.

Here, it is assumed that the user wears (or possesses) the measurement-facilitating device 100, but, for example, the environmental data acquisition unit 301 may acquire the position data of, for example, the wristwatch wearable terminal 120 worn by the user, and this position data may be used. In this case, the measurement-facilitating device 100 acquires the position data from the wristwatch wearable terminal 120 via the communication interface 201, and the environmental data acquisition unit 301 determines whether the user is indoors.

Because the position data is desired to be a place where the user is located, when the user wears the wristwatch wearable terminal 120, the wristwatch wearable terminal 120 is desirable rather than the measurement-facilitating device 100. However, in a case where the measurement-facilitating device 100 is possessed almost all the time or is always carried in, for example, a bag, there is no problem that the position data of the measurement-facilitating device 100 is determined to match the position data of the user. For example, since the position data also depends on whether the accuracy of the GPS reception unit is better, in a case where the accuracy of the measurement-facilitating device 100 is better than that of the wristwatch wearable terminal 120 and the measurement-facilitating device 100 is indoors or outdoors same as the user in most time, measuring the position data by the measurement-facilitating device 100 is desired.

### Step S402

In step S402, the control unit 210 operates as the environmental data acquisition unit 301, and acquires the indoor environmental data from an indoor sensor via the communication interface 201 by Bluetooth. Note that the communication interface 201 may receive data by another communication method other than Bluetooth, but may be another short-range wireless communication system.

### Step S403

In step S403, the control unit 210 operates as the environmental data acquisition unit 301, and acquires the outdoor environmental data from, for example, an outdoor sensor or the server 130 via the network 140. Step S404 In step S404, the control unit 210 operates as the user influence determination unit 303, and determines whether the environmental data acquired by the environmental data acquisition unit 301 adversely affects the biometric data of the user. When the user influence determination unit 303 determines that the biometric data is not adversely affected, the process returns to step S401, and when the user influence determination unit 303 determines that the biometric data is adversely affected, the process proceeds to step S405. The user influence determination unit 303 determines whether the environmental data acquired by the environmental data acquisition unit 301 meets the preliminarily stored condition regarding the environment set based on the biometric data of the user and indicating that the biometric data of the user is susceptible to the change. This condition is as described above.

### Step S405

In step S405, the control unit 210 operates as the instruction unit 304, and instructs the user to measure the biometric data determined to be adversely affected in the user influence determination unit 303 via the output device 204.

### Operation Example: Table Creation

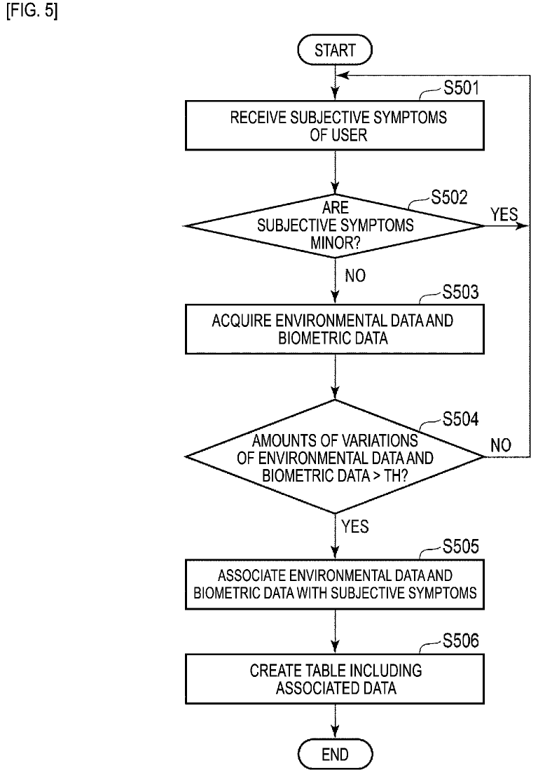

Next, an overview of the operations of the measurement-facilitating device 100 will be described with reference to FIG. 5.

FIG. 5 is a flowchart depicting an example of a processing procedure of the table creation regarding the measurement-facilitating device 100. Note that the processing procedure described below is merely an example, and each process may be changed to the extent possible. Further, in the processing procedure described below, steps can be omitted, substituted, and added in accordance with the embodiment as appropriate.

### Activation

First, the user starts the measurement-facilitating device 100 via the input device 203, and further receives inputs, such as a configuration. The control unit 210 in the measurement-facilitating device 100 advances processing according to the following processing procedure.

### Step S501

In step S501, the control unit 210 operates as the physical condition reception unit 305, and acquires the content related to the physical condition of the user (e.g., subjective symptoms of the user) via the input device 203.

### Step S502

In step S502, the control unit 210 operates as the physical condition reception unit 305 to determine whether the subjective symptoms, which are contents related to the physical condition of the user acquired in step S501, are minor. For example, the subjective symptoms are selected by questions to the user and the corresponding answers, for example, whether a consultation with a physician is required is determined. When the consultation with the physician is determined as unnecessary, the subjective symptoms are determined as minor, and when not, the subjective symptoms are determined as not minor. The determination of whether the subjective symptoms are minor may be determined by the user, not limited to this example, and many modified examples are possible. In a case where the physical condition reception unit 305 determines that the physical condition data is not minor, the physical condition reception unit 305 may cause the storage unit 202 to store the physical condition data.

In step S502, when the subjective symptoms are determined as minor, the process proceeds to step S501, and when the subjective symptoms are determined as not minor, the process proceeds to step S503.

### Step S503

In step S503, the control unit 210 operates as the environmental data acquisition unit 301 and the biometric data acquisition unit 302, acquires the environmental data and biometric data in conjunction with a period during which the user complains of subjective symptoms, and causes the storage unit 202 to store the data.

### Step S504

In step S504, the control unit 210 operates as the association unit 306 and associates the amount of variation of the measured environmental data with the amount of variation of the measured biometric data that varies in synchronization with the amount of variation of the measured environmental data. The control unit 210 determines whether the amounts of variation of the measured environmental data and the measured biometric data corresponding to the subjective symptoms of the user are greater than or equal to a threshold (also abbreviated as TH). Not the determination by the comparison with the threshold, but determination by a comparison whether the amount of variation is within a certain appropriate range may be made, and a determination criterion may be determined by the type and a property of the measured environmental data and/or the measured biometric data. For example, with the measured biometric data being the blood pressure data, the blood pressure value can be determined as an optimal blood pressure, a high blood pressure, or a low blood pressure according to the rage, and therefore determination with the range is desirable. Besides, with the measured biometric data being the electrocardiographic data, the determination is made by the shape of the electrocardiographic data in some cases. Since how extent the shape appears becomes a determination criterion, for example, the determination with a threshold is desirable in this case.

The association unit 306 sets the amount of variation of the associated measured environmental data that matches the amount of variation of the environmental data acquired by the environmental data acquisition unit 301 within a certain range as meeting the condition.

In step S504, when the respective amounts of variation of the measured environmental data and the measured biometric data are greater than the threshold, the process proceeds to step S505, and when not greater, the process returns to step S501.

### Step S505

In step S505, the control unit 210 operates as the association unit 306 and associates the measured environmental data corresponding to the subjective symptoms of the user and the measured biometric data with the subjective symptoms. The measured environmental data and the measured biometric data are associated with the subjective symptoms including the respective amounts of variation. The subjective symptoms may be ranked according to the symptoms and may be classified as, for example, a grave symptom and a slight symptom.

### Step S506

In step S506, the control unit 210 operates as the table creation unit 307, and creates the table including the data associated in step S505. The table may be stored by the table creation unit 307, or may be stored in a predetermined area in the storage unit 202, and the table creation unit 307 may access the table and provide the table to the user influence determination unit 303.

Based on the correlation between the type of biometric data, the measured environmental data, and the measured biometric data, the table creation unit 307 creates the table showing the correspondence with the measured environmental data that affects the biometric data of the user meeting the above-described condition. For example, in a case where the environmental data acquisition unit 301 acquires the environmental data similar to the measured environmental data included in the table, the user influence determination unit 303 determines that the acquired environmental data meets the condition, and the instruction unit 304 instructs the measurement of the corresponding biometric data shown by the table.

### Actions and Effects

As described above, the measurement-facilitating device 100 of the present embodiment can acquire the suitable environmental data from the indoor sensor or the outdoor sensor (and/or the server storing the outdoor data) depending on whether the user is indoors in step S401. In step S404, whether the amount of variation of the environmental data adversely affects the user is determined in step S404. When it is determined that the negative influence is given, the user is instructed to perform measurement and thus the biometric data according to the environmental data can be measured. Thus, by referring to the environmental data and the biometric data, the user can make use of the data to manage own health. In addition, the measurement-facilitating device 100 of the embodiment can recognize which environmental change should be taken care of by the user himself/herself in advance. In addition, the user is notified that the biometric data of the user is susceptible according to the environmental change, and this shows the user that which environment should be changed.

Whether the amount of variation of the environmental data gives the negative effect is determined and only the required biometric data can be measured. Therefore, a time used by the user for input and measurement can be minimized, it is expected that useful data are measured as the environmental data and the biometric data in most cases, wasteful data is reduced, and resources in the storage unit 202 can be effectively used. Whether the amount of variation of the environmental data gives the negative effect is determined, and then the biometric data is measured in the case where the amount of variation gives the negative effect. This allows eliminating unnecessary measurements, reducing the number of measurements of the biometric data by the wristwatch wearable terminal 120, and reducing a failure, a reduction in power supply, and wear of a cuff and an electrode of the wristwatch wearable terminal 120.

Furthermore, by the procedure depicted in FIG. 5, the table effective for the determination by the user influence determination unit 303 can be created, and the user influence determination unit 303 can appropriately determine whether there is an abnormality in variation in the biometric data of the user from the amount of variation of the environmental data. Additionally, when the useful data are accumulated in the storage unit 202 or the table creation unit 307 to be learned, the content of the table is more refined and the accuracy of the determination by the user influence determination unit 303 can be further increased.

### Modified Examples

While embodiments of the present invention have been described in detail above, the foregoing description is merely illustrative of the present invention in all respects. Of course, various modified examples and variations can be made without departing from the scope of the present invention. For example, the following changes are possible. Further, specific configurations according to the embodiment may be adopted as appropriate in the implementation of the invention. Note that, in the following, the same reference numerals are used for components that are the same as those of the above-described embodiment, and descriptions thereof are omitted as appropriate. The following modified examples can be combined as appropriate.

1

While an example of the processing procedure of FIG. 5 is processing in a case where the physical condition reception unit 305 receives the content of the physical condition, even in a case where the physical condition data is absent in the storage unit 202, the association unit 306 may associate the environmental data with the corresponding biometric data, and the table creation unit 307 may create the table associating the environmental data with the biometric data. For example, the table is created based on the environmental data and biometric data measured in the past. In addition, the user may determine whether the instruction to measure the biometric data from the instruction unit 304 is appropriate, the storage unit 202 stores the data input by the user via the input device 203 and store reliability of the association between the environmental data and the biometric data based on the data, and the table creation unit 307 may create the table based on the reliability. In addition, the user influence determination unit 303 is caused to refer to only the table in which the highly-reliable environmental data and biometric data are associated. Whether the reliability is high is determined, for example, when a percentage of the answer “reliable” from the user is greater than or equal to a predetermined value, it is determined that the reliability is high. In addition, for example, the subsequent medical history of the user may be fed back to determine reliability from the association between the biometric data and the disease condition (in a case where the biometric data that gets worse due to the variation is associated with the disease condition, the reliability is determined as high).

2

Although not illustrated, the wristwatch wearable terminal 120 and/or the measurement-facilitating device 100 may include an acceleration sensor, a pressure sensor, a gyro sensor, and/or a geomagnetic sensor.

The acceleration sensor is a sensor that detects acceleration, for example, a three-axis acceleration sensor, and detects an acceleration of the sensor with respect to three axes that are linearly independent (for example, three axes orthogonal to each other). The acceleration sensor outputs an acceleration signal indicating acceleration in three directions to the control unit 210. The acceleration sensor can acquire a roll angle and a pitch angle from the value of acceleration during rest.

The pressure sensor is general one to detect the pressure and, for example, measurement of the air pressure by the pressure sensor allows detecting an altitude of the user.

A gyro sensor is general one that can detect an angular velocity of the sensor, and for example, is a three-axis gyro sensor that detects an angular velocity of the sensor with respect to three axes that are linearly independent. The gyro sensor outputs angular velocity signals representing the angular velocities in three directions to the control unit 210.

The geomagnetic sensor is general one and used to determine an attitude of the user. The geomagnetic sensor is, for example, a three-axis geomagnetic sensor that detects an intensity of geomagnetism around the sensor with respect to three axes including a direction and a strength (a magnitude).

The control unit 210 may use a technique for correcting an error due to a drift for the angular velocity, which is obtained by the gyro sensor, using the information from the acceleration sensor and the geomagnetic sensor, may obtain the angle by integrating the angular velocity from initial attitude information of the user, and may obtain an attitude angle of each sensor at a desired elapsed time from the initial time. As the initial attitude, a roll angle and a pitch angle can be obtained by the acceleration sensor. A yaw angle can be obtained using the geomagnetic sensor.

The control unit 210 calculates a three-dimensional component of the magnetic field with the inclination error corrected from the three-dimensional component of the magnetic field obtained by the geomagnetic sensor and the roll angle and the pitch angle obtained previously. The yaw angle can be calculated from the x component and the y component among the three-dimensional component of the magnetic field with the inclination error has been corrected. For example, when the user moves from the initial attitude, time integration is performed on the angular velocity to obtain the angle, and an attitude angle of each sensor at any given time can be obtained from the initial attitude and the elapsed time.

<3>

The device of the present invention may also be realized by a computer and a program, and the program may be recorded in a recording medium (or storage medium), or provided through a network.

Further, the above-described devices and the device portions thereof can each be implemented by either a hardware configuration or a combined configuration of a hardware resource and software. As the software of the combined configuration, a program that is installed in a computer from a network or a computer-readable recording medium (or storage medium) in advance, and executed by a processor of the computer to achieve the operations (or the functions) of the respective devices by the computer is used.

<4>

Note that this invention is not limited to the embodiments described above, and can be embodied by modifying the components in an implementation stage without departing from the gist thereof. Additionally, various inventions can be formed by appropriately combining a plurality of components disclosed in the embodiments described above. For example, some components may be omitted from all the components described in the embodiments. Further, the components of the different embodiments may be combined appropriately.

<5>

Further, “and/or” means any one or more items of the items connected and listed by the “and/or” statement. As a specific example, “x and/or y” means any element of the set of three elements {(x), (y), (x, y)}. As another one specific example, “x, y, and/or z” means any element among the set of seven elements {(x), (y), (z), (x, y), (x, z), (y, z), (x, y, z)}.

### Supplementary Note 1

The measurement-facilitating device (100) includes the first acquisition unit (301) that acquires the environmental data regarding the external environment of the user, the first determination unit (303, 302, 305, 306, 307) that determines whether the environmental data meets the preliminarily stored condition regarding the environment set based on the biometric data of the user and indicating that the biometric data of the user is susceptible to the change, and the instruction unit (304) that instructs the measurement of the biometric data when the environmental data is determined to meet the condition.

### REFERENCE SIGNS LIST

100 Measurement device
120 Wristwatch wearable terminal
130 Server
140 Network
150 GPS satellite
160 Sensor
201 Communication interface
202 Storage unit
203 Input device
204 Output device
205 Clocking device
206 Power source unit
207 External interface
208 GPS reception unit
210 Control unit
301 Environmental data acquisition unit
302 Biometric data acquisition unit
303 User influence determination unit
304 Instruction unit
305 Physical condition reception unit
306 Association unit
307 Table creation unit

1. A measurement-facilitating device comprising:
one or more processors configured to:
acquire environmental data regarding an external environment of a user;
determine whether the environmental data meets a preliminarily stored condition regarding an environment set based on biometric data of the user and indicating that the biometric data of the user is susceptible to a change; and
instruct measurement of the biometric data when the environmental data is determined to meet the condition.

2. The measurement-facilitating device according to claim 1, wherein the one or more processors are further configured to:
calculate the condition based on a correlation between the measured environmental data and the measured biometric data.

3. The measurement-facilitating device according to claim 2, wherein the one or more processors are further configured to:
associate an amount of variation of the measured environmental data with an amount of variation of the measured biometric data that varies in synchronization with the amount of variation of the measured environmental data; and
set the amount of variation of the associated measured environmental data matching the amount of variation of the environmental data that is acquired within a certain range as meeting the condition.

4. The measurement-facilitating device according to claim 3, wherein the one or more processors are further configured to:
receive a content of a change in a physical condition that the user is aware of, and
associate the amount of variation of the measured environmental data, the content of the change in the physical condition, and the amount of variation of the measured biometric data based on the content of the change in the physical condition and the amount of variation of the measured biometric data.

5. The measurement-facilitating device according to claim 1, wherein the one or more processors are further configured to:
acquire a table indicating a correspondence with the measured environmental data meeting the condition based on a type of the biometric data and a correlation between the measured environmental data and the measured biometric data, and
refer to the table to determine whether the environmental data that is acquired meets the condition.

6. The measurement-facilitating device according to claim 5, wherein
the table is determined for each of the measured environmental data and for each user based on an amount of variation of the measured environmental data and an amount of variation of the measured biometric data.

7. The measurement-facilitating device according to claim 1, wherein the one or more processors are further configured to:
determine whether the user is indoors, and
change the acquired environmental data by whether the user is outdoors or indoors.

8. The measurement-facilitating device according to claim 7, wherein
when the user is determined to be indoors, the one or more processors are further configured to acquire the environmental data from a sensor installed indoors based on a near-field wireless communication, preferentially employ the indoor environmental data over the outdoor environmental data, and determine whether the biometric data of the user is susceptible to a change in an environment.

9. The measurement-facilitating device according to claim 1, wherein
when the one or more processors determine that the acquired environmental data meets the condition, the one or more processors are further configured to notify the user of an alert indicating that the biometric data is likely to change by an environment.

10. A measurement-facilitating method comprising:
acquiring environmental data regarding an external environment of a user;
determining whether the environmental data meets a preliminarily stored condition regarding an environment set based on biometric data of the user and indicating that the biometric data of the user is susceptible to a change; and
instructing measurement of the biometric data when the environmental data is determined to meet the condition.

11. A non-transitory recording medium that records a program for causing a computer to execute functions in the measurement-facilitating device according to claim 1.
